(19) United States
(12) Patent Application Publication
(10) Pub. No.: US 2010/0125325 A1
(43) Pub. Date: May 20, 2010

(54) Stent With Cathodic Protection and Stent Delivery System

(75) Inventors: Jeffrey Allen, Santa Rosa, CA (US); Michael Krivoruchko, Forestville, CA (US); Joseph Berglund, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/274,848

(22) Filed: Nov. 20, 2008

(51) Int. Cl. A61F 2/06; A61M 29/00

(52) U.S. Cl. 623/1.11; 623/1.15; 604/96.01

(57) ABSTRACT

The stent with cathodic protection and stent delivery system includes a stent delivery system including a catheter; a balloon operably attached to the catheter; and a stent disposed on the balloon. The stent includes a stent body having a first stent layer of an anodic stent material disposed about a second stent layer of a cathodic stent material; and a battery having a first battery layer of an anodic battery material and a second battery layer of a cathodic battery material. The first stent layer is electrically coupled to the first battery layer and the second stent layer is electrically coupled to the second battery layer.

### TECHNICAL FIELD

The technical field of this disclosure is medical implant devices, particularly, stents with cathodic protection.

### BACKGROUND OF THE INVENTION

Stents are generally cylindrical shaped devices that are radially expandable to hold open a segment of a blood vessel or other anatomical lumen after implantation into the body lumen. Stents have been developed with coatings to deliver drugs or other therapeutic agents.

Stents are used in conjunction with balloon catheters in a variety of medical therapeutic applications including intravascular angioplasty. For example, a balloon catheter device is inflated during PTCA (percutaneous transluminal coronary angioplasty) to dilate a stenotic blood vessel. The stenosis may be the result of a lesion such as a plaque or thrombus. After inflation, the pressurized balloon exerts a compressive force on the lesion thereby increasing the inner diameter of the affected vessel. The increased interior vessel diameter facilitates improved blood flow. Soon after the procedure, however, a significant proportion of treated vessels re-narrow.

To prevent restenosis, short flexible cylinders, or stents, constructed of metal or various polymers are implanted within the vessel to maintain lumen size. The stents acts as a scaffold to support the lumen in an open position. Various configurations of stents include a cylindrical tube defined by a mesh, interconnected stents or like segments. Some exemplary stents are disclosed in U.S. Pat. No. 5,292,331 to Boneau, U.S. Pat. No. 6,090,127 to Globerman, U.S. Pat. No. 5,133,732 to Wiktor, U.S. Pat. No. 4,739,762 to Palmaz and U.S. Pat. No. 5,421,955 to Lau. Balloon-expandable stents are mounted on a collapsed balloon at a diameter smaller than when the stents are deployed. Stents can also be self-expanding, growing to a final diameter when deployed without mechanical assistance from a balloon or like device.

Concern over the long-term effects of stents in the body has led to experimentation with bioabsorbable stents, i.e., stents that are absorbed by the body after deployment. Materials used for bioabsorbable stents have included bioabsorbable metals, such as highly reactive, corrodible magnesium. Unfortunately, the materials used to date have failed to produce satisfactory results. A bioabsorbable stent needs to seal any dissection and provide scaffolding to prevent wall recoil until such scaffolding is no longer needed. A bioabsorbable stent made of bare magnesium lasts a few weeks after deployment in a vessel, but should be present for several months to prevent wall recoil. With the stent gone prematurely, the vessel is reduced in diameter, making the treatment ineffective.

It would be desirable to have a stent with cathodic protection that would overcome the above disadvantages.

### SUMMARY OF THE INVENTION

One aspect of the present invention provides a stent delivery system including a catheter; a balloon operably attached to the catheter; and a stent disposed on the balloon. The stent includes a stent body having a first stent layer of an anodic stent material disposed about a second stent layer of a cathodic stent material; and a battery having a first battery layer of an anodic battery material and a second battery layer of a cathodic battery material. The first stent layer is electrically coupled to the first battery layer and the second stent layer is electrically coupled to the second battery layer.

Another aspect of the present invention provides a stent including a stent body having a first stent layer of an anodic stent material disposed about a second stent layer of a cathodic stent material; and a battery having a first battery layer of an anodic battery material and a second battery layer of a cathodic battery material. The first stent layer is electrically coupled to the first battery layer and the second stent layer is electrically coupled to the second battery layer.

Another aspect of the present invention provides a stent including a stent body having a first stent layer of an anodic stent material disposed about a second stent layer of a cathodic stent material; and means for establishing an electrical potential between the first stent layer and the second stent layer.

The foregoing and other features and advantages of the invention will become further apparent from the following detailed description of the presently preferred embodiments, read in conjunction with the accompanying drawings. The detailed description and drawings are merely illustrative of the invention, rather than limiting the scope of the invention being defined by the appended claims and equivalents thereof.

### BRIEF DESCRIPTION OF THE DRAWINGS

FIG. 1 is a perspective view of a stent delivery system made in accordance with the present invention.

FIG. 2 is a side view of a stent with cathodic protection made in accordance with the present invention.

FIG. 3 is a schematic diagram of a stent with cathodic protection made in accordance with the present invention.

FIG. 4 is a detail view of another embodiment of a stent with cathodic protection made in accordance with the present invention.

### DETAILED DESCRIPTION OF PRESENTLY PREFERRED EMBODIMENTS

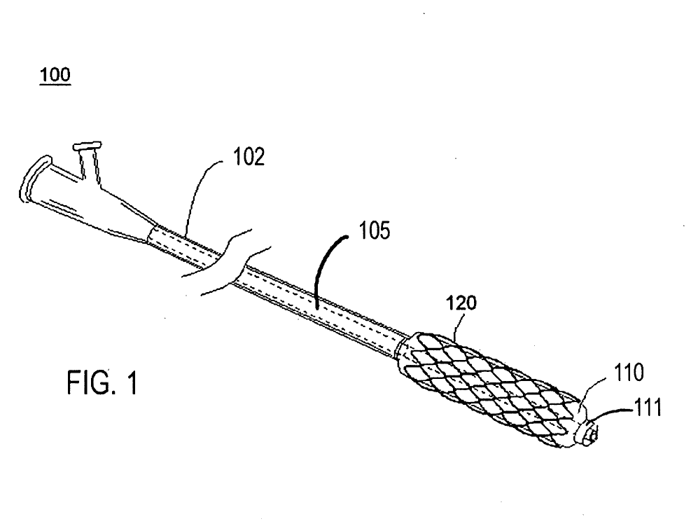

FIG. 1 is a perspective view of a stent delivery system made in accordance with the present invention. The stent delivery system 100 includes a catheter 105, a balloon 110 operably attached to the catheter 105, and a stent 120 disposed on the balloon 110. The balloon 110, shown in an inflated state, can be any variety of balloons capable of expanding the stent 120. The balloon 110 can be manufactured from a material such as polyethylene, polyethylene terephthalate (PET), nylon, Pebax® polyether-block co-polyamide polymers, or the like. In one embodiment, the stent delivery system 100 can include retention means 111, such as mechanical or adhesive structures, for retaining the stent 120 on the balloon 110 until the stent 120 is deployed. The catheter 105 may be any variety of balloon catheters, such as a PTCA (percutaneous transluminal coronary angioplasty) balloon catheter, capable of supporting a balloon during angioplasty. The stent delivery system 100 can also include a sheath 102 through which the stent 120 is delivered to the deployment site.

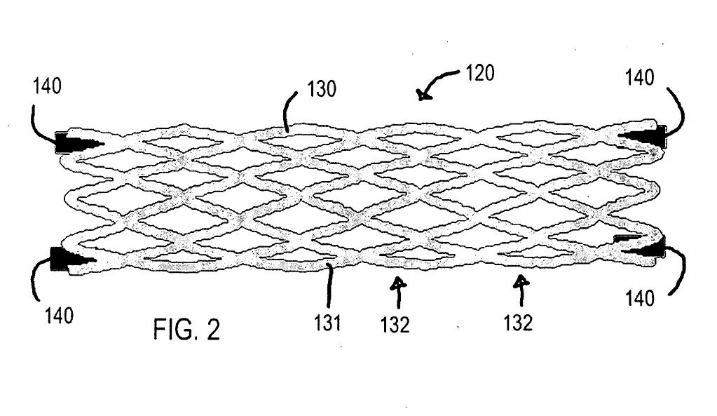

FIG. 2 is a side view of a stent with cathodic protection made in accordance with the present invention. In this embodiment, the battery is external to the stent body to provide cathodic protection.

The stent 120 includes a stent body 130 and at least one battery 140 electrically coupled to the stent body 130. The stent body 130 includes a number of stent body segments 132 made of stent segments 131. The pattern of the stent body segments 132 can be W-shaped or can be a more complex shape with the elements of one segment continuing into the adjacent segment. The stent 120 can be installed in the stent delivery system of FIG. 1 for implantation in a body lumen.

Referring to FIG. 2, the stent body 130 is conventional to stents generally and can be made of a wide variety of medical implantable materials. In one embodiment, the stent body 130 is bioabsorbable. The stent body 130 has a first stent layer of an anodic stent material disposed about and enclosing a second stent layer of a cathodic stent material. The stent body 130 can be welded, laser cut, molded, or consist of filaments or fibers which are wound or braided together in order to form a continuous structure. Depending on the material, the stent can be self-expanding, or be expanded by a balloon or some other device. In one embodiment, the stent body 130 can carry a coating, such as a polymer coating carrying one or more therapeutic agents, such as anti-inflammatory agents or anti-proliferative agents. In another embodiment, the stent body 130 can include one or more therapeutic agents within the stent material.

The battery 140 has a first battery layer of an anodic battery material and a second battery layer of a cathodic battery material to develop an electrical potential. The first stent layer of the stent body 130 is electrically coupled to the first battery layer and the second stent layer of the stent body 130 is electrically coupled to the second battery layer. The electrical potential prevents corrosion of the first stent layer until the battery 140 is depleted. An external battery 140 can be attached to the stent body 130 with welding, brazing, or the like, in the same manner of attachment as for the marker bands used to make points on the stent visible during fluoroscopy. In this embodiment, the battery is external to the stent body, i.e., the battery 140 is outside of the stent body 130.

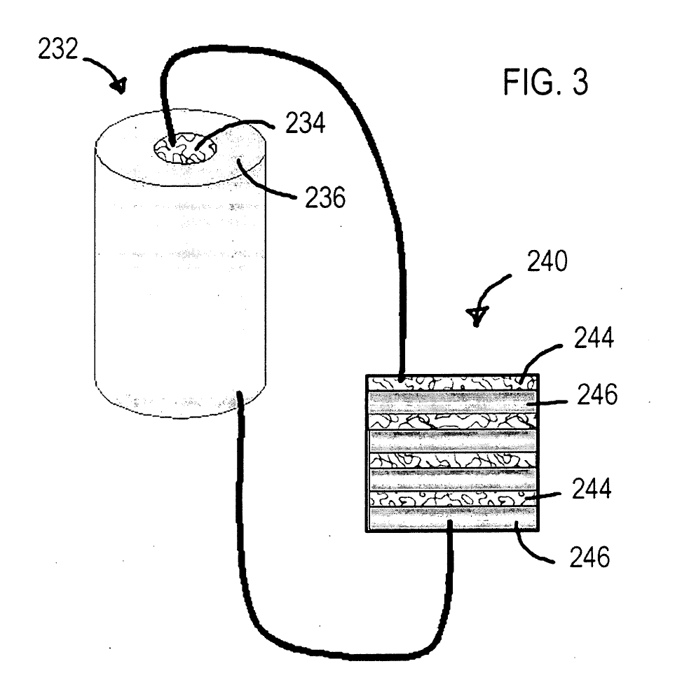

FIG. 3 is a schematic diagram of a stent with cathodic protection made in accordance with the present invention. A battery provides electrical potential at the stent body to prevent corrosion of the stent body by reversing the corrosion potential normally present when the stent is deployed in a vessel.

The strut segment 232 of the stent body in this example has a first stent layer 236 of an anodic stent material disposed about a second stent layer 234 of a cathodic stent material. The battery 240 has a number of first battery layers 246 of an anodic battery material alternating with a number of second battery layers 244 of a cathodic battery material. The outermost first battery layer 246 is electrically coupled to the first stent layer 236 and the outermost second battery layer 244 is electrically coupled to the second stent layer 234. Thus, the cathodic battery material is electrically coupled to the cathodic stent material and the anodic battery material is electrically coupled to the anodic stent material. The cathodic materials are dissimilar from the anodic materials, so an electrical potential is generated when the materials are in contact. The electrical potential reverses the corrosion potential normally present when the stent is deployed in a vessel to provide cathodic protection.

The stent material and battery material can be selected as desired for a particular application. In one embodiment, the anodic stent material is the same as the anodic battery material and the cathodic stent material is the same as the cathodic battery material. In one embodiment, the anodic stent material and/or the anodic battery material is magnesium or a magnesium alloy (such as WE43 magnesium alloy). In one embodiment, the cathodic stent material and/or the cathodic battery material is iron, stainless steel (such as 316 stainless steel), copper, gold, or platinum. Gold or platinum in the battery 240 can also serve as marker bands to make points on the stent show up during fluoroscopy.

The battery 240 provides impressed current cathodic protection. In operation, the second stent layer 234 provides cathodic protection for the first stent layer 236, which is exposed to fluid in the lumen in which the stent is implanted and subject to corrosion. The first stent layer 236 is protected until the sacrificial cathode second stent layer 234 is depleted. When the second stent layer 234 is depleted, there is no longer cathodic protection and the first stent layer 236 will corrode. The thickness of the second stent layer 234 and/or the amount of the cathodic stent material in the second stent layer 234 can be selected to determine a depletion time for the second stent layer 234. As defined herein, the depletion time is the time at which cathodic protection is no longer provided by the second stent layer. When the first stent layer 236 is magnesium, the corrosion products are absorbed in the body.

The battery 240 can have the number of first battery layers 246 and second battery layers 244 required to provide a desired electrical potential. A number of batteries can be electrically coupled in series or parallel to provide the desired electrical potential or capacity, respectively. In one embodiment, the battery layers can be fabricated by depositing the layers using sputtering, vapor deposition, or the like.

The dual layer strut segment 232 of the stent body in this example includes a single second stent layer 234 having a circular cross section and a single first stent layer 236 having an annular cross section. The second stent layer 234 acts as a sacrificial cathode. Those skilled in the art will appreciate that the strut segments making up the stent body can have any cross section desired for a particular application. In another embodiment, the stent layers can have rectangular cross sections. In one embodiment, the strut segment 232 of the stent body can be fabricated by depositing layers using sputtering, vapor deposition, or the like. In another embodiment, the strut segment 232 of the stent body can be fabricated by co-extrusion of the anodic stent material and cathodic stent material into the stent layers.

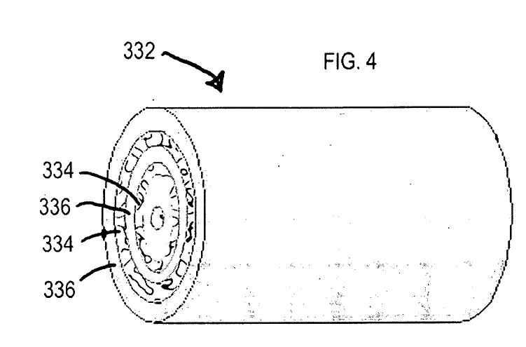

FIG. 4 is a detail view of another embodiment of a stent with cathodic protection made in accordance with the present invention. The multi-layer strut segment 332 of the stent body in this example has a number of first stent layers 336 of an anodic stent material alternating with a number of second stent layers 334 of a cathodic stent material. The outermost first stent layer 336 can be electrically coupled to the first battery layer made of anodic battery material and the central innermost second stent layer 334 can be electrically coupled to the second battery layer made of cathodic battery material.

The strut segment 332 of the stent body can have the number of second stent layers 334 and first stent layers 336. Those skilled in the art will appreciate that the strut segments making up the stent body can have any cross section desired for a particular application. In another embodiment, the stent layers can have rectangular cross sections. In one embodiment, the strut segment 332 of the stent body can be fabricated by depositing layers using sputtering, vapor deposition, or the like. In another embodiment, the strut segment 332 of the stent body can be fabricated by co-extrusion of the anodic stent material and cathodic stent material into the stent layers.

The strut segment 332 of the stent body can be used as a battery integral to the stent body. In one embodiment, the whole stent body can be made of strut segments 332 with internal electrical coupling as desired for a particular application. In another embodiment, the stent body can include a number of strut segments 332 interspersed with dual layer strut segments 232 as illustrated in FIG. 3. In one example, one or more multi-layer strut segment can be integrated as part of each stent body segment.

It is important to note that FIGS. 1-4 illustrate specific applications and embodiments of the present invention, and are not intended to limit the scope of the present disclosure or claims to that which is presented therein. Upon reading the specification and reviewing the drawings hereof, it will become immediately obvious to those skilled in the art that myriad other embodiments of the present invention are possible, and that such embodiments are contemplated and fall within the scope of the presently claimed invention.

While the embodiments of the invention disclosed herein are presently considered to be preferred, various changes and modifications can be made without departing from the spirit and scope of the invention. The scope of the invention is indicated in the appended claims, and all changes that come within the meaning and range of equivalents are intended to be embraced therein.

1. A stent delivery system comprising:
a catheter;
a balloon operably attached to the catheter; and
a stent disposed on the balloon;
wherein the stent comprises:
a stent body, the stent body having a first stent layer of an anodic stent material disposed about a second stent layer of a cathodic stent material; and
a battery, the battery having a first battery layer of an anodic battery material and a second battery layer of a cathodic battery material;
wherein the first stent layer is electrically coupled to the first battery layer and the second stent layer is electrically coupled to the second battery layer.

2. The stent delivery system of claim 1 wherein the anodic stent material is the same as the anodic battery material and the cathodic stent material is the same as the cathodic battery material.

3. The stent delivery system of claim 1 wherein the anodic stent material is selected from the group consisting of magnesium and magnesium alloys.

4. The stent delivery system of claim 1 wherein the cathodic stent material is selected from the group consisting of iron, stainless steel, copper, gold, and platinum.

5. The stent delivery system of claim 1 wherein the battery is external to the stent body.

6. The stent delivery system of claim 1 wherein the battery is integral to the stent body.

7. The stent delivery system of claim 1 wherein the stent body further comprises a plurality of anodic stent layers of the anodic stent material alternating with a plurality of cathodic stent layers of the cathodic stent material.

8. The stent delivery system of claim 1 wherein the battery further comprises a plurality of anodic battery layers of the anodic battery material alternating with a plurality of cathodic battery layers of the cathodic battery material.

9. The stent delivery system of claim 1 wherein thickness of the second stent layer is selected to determine a depletion time of the second stent layer.

10. The stent delivery system of claim 1 wherein an amount of the cathodic stent material is selected to determine a depletion time of the second stent layer.

11. A stent comprising:
a stent body, the stent body having a first stent layer of an anodic stent material disposed about a second stent layer of a cathodic stent material; and
a battery, the battery having a first battery layer of an anodic battery material and a second battery layer of a cathodic battery material;
wherein the first stent layer is electrically coupled to the first battery layer and the second stent layer is electrically coupled to the second battery layer.

12. The stent of claim 11 wherein the anodic stent material is the same as the anodic battery material and the cathodic stent material is the same as the cathodic battery material.

13. The stent of claim 11 wherein the anodic stent material is selected from the group consisting of magnesium and magnesium alloys.

14. The stent of claim 11 wherein the cathodic stent material is selected from the group consisting of iron, stainless steel, copper, gold, and platinum.

15. The stent of claim 11 wherein the battery is external to the stent body.

16. The stent of claim 11 wherein the battery is integral to the stent body.

17. The stent of claim 11 wherein the stent body further comprises a plurality of anodic stent layers of the anodic stent material alternating with a plurality of cathodic stent layers of the cathodic stent material.

18. The stent of claim 11 wherein the battery further comprises a plurality of anodic battery layers of the anodic battery material alternating with a plurality of cathodic battery layers of the cathodic battery material.

19. The stent of claim 11 wherein thickness of the second stent layer is selected to determine a depletion time of the second stent layer.

20. The stent of claim 11 wherein an amount of the cathodic stent material is selected to determine a depletion time of the second stent layer.

21. A stent comprising:
a stent body, the stent body having a first stent layer of an anodic stent material disposed about a second stent layer of a cathodic stent material; and
means for establishing an electrical potential between the first stent layer and the second stent layer.
